(19) United States
(12) Patent Application Publication
(10) Pub. No.: US 2009/0238885 A1
(43) Pub. Date: Sep. 24, 2009

(54) PROTEIN ENCAPSULATED PARTICLES

(75) Inventors: Aart Cornelis Alting, Ede (NL); Theodorus Arnoldus Gerardus Floris, Arnhem (NL); Fanny Chantal Jacqueline Weinbreck, Amersfoort (NL); Jeroen Grandia, Ede (NL)

Correspondence Address: FOLEY AND LARDNER LLP;SUITE 500, 3000 K STREET NW, WASHINGTON, DC, 20007, US

(73) Assignee: Nizo Food research B.V.

(21) Appl. No.: 12/301,969

(22) PCT Filed: May 21, 2007
(86) PCT No.: PCT/NL2007/050233

§ 371 Date: Apr. 30, 2009

(30) Foreign Application Priority Data
May 22, 2006 (EP) 06114320.2

(51) Int. Cl. A61K 9/14; B01J 13/04; A23L 1/22; A23P 1/04; A61P 43/00

(52) U.S. Cl. 424/491; 264/4.1; 426/471; 427/213.3; 428/402.2; 428/402.24; 426/656; 426/580; 426/590; 426/589; 426/565; 264/4.7

(57) ABSTRACT

Particles that are encapsulated by protein are provided. Also a method for preparing protein encapsulated particles involving spraying and drying of an activated protein solution is provided. The protein encapsulated particles are particularly suited for food, feed, cosmetic and pharma applications.

### FIELD OF THE INVENTION

The present invention concerns a method for making encapsulated particles, which encapsulate either air, core material or fat droplets by protein based encapsulating agents. The encapsulated particles thus provided are suitable ingredients for various products, in particular food products.

### BACKGROUND OF THE INVENTION

The use of encapsulated ingredients in various products is widely known. In particular encapsulation techniques have been developed to protect the high quality and stability of ingredients in food, health and medical products. To this end encapsulation agents have been developed to meet the criteria of successfully providing long term stability and protection against deteriorating factors.

Acceptable encapsulating agents must be safe and non-hazardous to the consumer's health. For food products it should have a bland or no flavor. Besides protecting the encapsulated product from external factors such as oxygen, water, light or other compounds possibly causing deterioration, it should delay the release of an active ingredient pending its use.

Suitable encapsulation agents for food applications include natural gums, carbohydrates, fats and waxes and some proteins. Whereas gum arabic is one of the most widely used encapsulation agent in food applications the use of proteins is limited. The main protein that has been evaluated for encapsulation is gelatin. Gelatin has been successfully applied as encapsulation agent in the pharmaceutical industry, however, due to the high viscosity, even of solutions that are low in gelatin concentration, gelatin has limited use in spray-drying processes.

U.S. Pat. No. 5,601,760 describes a method for microencapsulation of a volatile or a non-volatile core material in an encapsulation agent consisting essentially of a whey protein. It is described that whey protein isolate and whey protein concentrate, optionally in combination with milk-derived or non-milk derived carbohydrates, and also β-lactoglobulin and mixtures of β-lactoglobulin and α-lactalbumin were used in a spray-drying encapsulation process. The resulting encapsulates were said to protect the core against deterioration by oxygen or from detrimental of other compounds or materials, to limit the evaporation or losses of volatile core materials and to release the core upon full hydration reconstitution. One example describes encapsulation of anhydrous milk fat in whey protein isolate that has been heated at 80° C. for 30 minutes. This treatment results in denaturation of whey proteins.

EP 1042960 describes a cappuccino creamer with advantageous foaming properties. The creamer is prepared by spray-drying a slurry that includes as essential constituents protein, lipid and carrier. The lipid includes dairy fats and vegetable oils. Suitable carriers include gum arabic and water soluble carbohydrates such as maltodextrin and lactose. The protein is partly denatured whey protein (concentrate or isolate). The product is said to contain buoyant, hydrated, insoluble, non-colloidal, irregularly shaped whey protein particles of approximately 10-200 microns in size, with an average particle size of about 60 microns. To provide coffee whitening and creamy mouth feel a significant amount of encapsulated fat has to be included.

U.S. Pat. No. 6,841,181 B2 describes the encapsulation of active food components using spray-drying technology. The process consists of mixing active ingredients with non-activated proteins and polysaccharides which are spray-dried to form a capsule. The capsules are 1-200 μm and up to 90% core material.

However, methods for making encapsulated food-grade particles having predefined properties are still needed, as the prior art methods cannot fully control the particle size, particle content, particle uniformity, water solubility/insolubility, etc.

It is an object of the invention to provide a method for producing encapsulated particles, whereby the production method allows to control the properties of the resulting particles.

### SUMMARY OF THE INVENTION

Contrary to the disclosures in U.S. Pat. No. 5,601,760 and EP 1042960, the present inventors found that proteins can be activated by specific activation treatments, so that protein particles with a certain minimal reactivity are obtained. This activation is a different principle than protein denaturation and activation is crucial for the formation of disulphide cross-links between activated protein aggregates during the drying step of the present invention. Activation can herein be achieved by various methods, such as heating, high pressure treatment etc. The resulting protein reactivity is determined by the overall treatment conditions (shear, protein concentration, type of protein, protein composition, type and concentration of salts, pH, other ingredients such as sugars and polysaccharides, fats). Thus, for example when heat treatment is used for activation, heating time and temperature (i.e. the two parameters commonly used to denature proteins) may influence reactivity, but are not the parameters that only determine reactivity. Because the combined overall conditions during treatment (such as heating) greatly influence reactivity, it is essential to the invention to determine the reactivity after activation treatment, so that one can use protein aggregates having a minimal reactivity in the spraying and drying process.

Thus, an essential step of the present method is to create protein aggregates having a minimum reactivity. The flexibility and control of the method allows three types of encapsulated particles to be made: protein encapsulated air particles, protein encapsulated core particles and protein encapsulated fat droplets. The particle size and properties can be controlled. These particles are particularly useful as ingredients in food and feed products or cosmetic products.

The present inventors have found a method for the preparation of protein encapsulated particles. The particles obtained are preferably as good as spherical. Also preferably essentially all particles obtained are spherical. The method involves an activation step (selected from one or more of e.g. a heating step, submission to pressure, etc.) of a protein solution to such an extent that protein aggregates having a minimum reactivity are formed. The activated protein aggregates are then sprayed to form particles, preferably essentially spherical particles, followed by drying of the particles. As mentioned, it is important that the protein solution that is used for spraying comprises sufficiently activated protein aggregates. In order to achieve this the starting protein in the solution to be activated should contain a sufficient proportion of activated groups. Therefore it is required that the starting protein is treated to such an extent and under such overall conditions (pH, concentration, shear, etc.) that the protein aggregates formed have a reactivity of at least 0.10 mM thiol or sulphydryl groups per 2 wt % protein solution, more preferably at least 0.15 mM thiol groups per 2 wt % protein solution, or more, as can be determined using Ellman's assay (Ellman, G. L. Tissue sulfhydryl groups. Arch. Biochem. Biophys. 1959, 82, 70-77).

Thus the invention concerns a method for the production of protein encapsulated particles, said method comprising
a. providing an aqueous solution comprising protein, most preferably food-grade protein as described below, and optionally mixing said solution with one or more additives (including e.g. one or more sensitive additives as described below),
b. submitting said aqueous solution, comprising said protein to an activation treatment (preferably a heat treatment, pressure treatment, etc.), to obtain activated protein aggregates having a reactivity of at least 0.10 mM sulfhydryl or thiol groups per 2 wt % protein solution, and optionally mixing said solution with one or more additives (including sensitive additives), and
c. spraying said treated aqueous solution, comprising said activated protein aggregates (and optionally said additives, preferably sensitive additives) to form particles and
d. drying said particles/sprayed protein coating.

Optionally further layers are added around the particles, as described herein below, to form multi-layered particles.

Also provided are particles obtainable by the method. The protein coating formed by such particles has unique properties, as do the particles themselves. For example, when air is coated using activated protein aggregates, very small (with a diameter of less than 50 μm), water-insoluble particles can be formed. The stable protein matrix of these particles is largely broken down after ingestion and can thus be suitably added to food products, such as drinks, soups, ice-cream etc.

Similarly, a very strong protein coating can be formed around a core material, so that the protein coating is only degraded in the gut, releasing the core or other components (additives) enclosed within the protein coating, or on or within the core. Further, the addition of one or more extra layers, such as further layers of activated protein aggregate or of other hydrocolloids (e.g. alginate, gum Arabic) results in particles that can only be degraded in the gut.

Further food or feed compositions, pharmaceutical or cosmetic compositions comprising a suitable amount of the particles of the invention are provided.

### GENERAL DEFINITIONS

“Food” refers herein to solid, liquid or semi-solid compositions suitable for human consumption or ingestion. The term thus encompasses beverages.

“Feed” refers herein to solid, liquid or semi-solid compositions suitable for consumption or ingestion by animals, especially domesticated animals such as pets or farm animals, especially mammals.

“Cosmetic compositions” refers to compositions for external use, such as skin lotions, creams, make-up, etc.

“Food grade” refers to components which are generally regarded as safe for human or animal consumption or ingestion.

“Probiotics” or “probiotic strain(s)” refers to strains of live micro-organisms, preferably bacteria, which have a beneficial effect on the host when ingested (e.g. enterally or by inhalation) by a subject.

The term “essentially spherical” or “as good as spherical” means that when observing the particles under a (light) microscope they appear to be round, hence not irregularly shaped.

In the context of this invention the phrase ‘essentially all particles are spherical’ means at least 60%, more preferably at least 70%, 80%, 90%, 95%, 98% or more of the particles are essentially spherical.

“Protein” refers herein to both full length proteins (as found naturally) and/or peptides, i.e. less than full-length amino acid sequences, i.e. fragments of proteins.

“Protein hydrolysates” refers to a mixture of proteins and/or peptides obtainable by (partial) breakage of peptide bonds, e.g. through enzymatic hydrolysis or other treatments.

“Water-insoluble particles” means that less than 50 wt. %, preferably 1-40 wt %, more preferably 2-30 wt % of the particles dissolves when dispersed in water at low pH (e.g. pH 2) under continuous stirring for 10 minutes, said water having a temperature of 37° C. and having been prepared by adding HCl to distilled water. Most preferably, less than 30 wt % is soluble in water at pH 3 under continuous stirring for 10 minutes.

The term “comprising” is to be interpreted as specifying the presence of the stated parts, steps or components, but does not exclude the presence of one or more additional parts, steps or components.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The term “sensitive additives” comprises additives which benefit from being protected from the environment (especially from the digestive tract or parts thereof, but also light, temperature, acids, radiation, etc.) and includes e.g. flavours, salts, enzymes, microorganisms (e.g. bacteria such as one or more probiotic bacterial strains), prebiotics, peptides, minerals, vitamins, fatty acids (e.g. PUFAs), drugs, bioactive components, hormones, etc.

### DETAILED DESCRIPTION OF THE INVENTION

### Methods According to the Invention

In one embodiment the invention provides a method for the production of protein encapsulated particles, said method comprising
a. providing an aqueous solution comprising protein, and optionally adding one or more additives to said solution (including e.g. one or more sensitive additives);
b. submitting said aqueous solution to an activation treatment to obtain activated protein aggregates having a reactivity of at least 0.10 mM sulphydryl or thiol groups per 2 wt % protein solution, as determined using the Ellman's assay, and optionally adding one or more additives to the solution comprising said activated protein aggregates;
c. spraying said solution, comprising said activated protein aggregates, to form particles, and
d. drying said particles.

Optionally further layers are added around the dried particles obtained in step d.

### Step a)

In step a) of the method a protein, most preferably a food-grade protein is dissolved in an aqueous solution, such as for example water. Preferably whole (essentially intact/full-length) proteins are used, although in certain embodiments also peptides, or hydrolyzed or partially hydrolyzed proteins and/or peptides may be used. Suitable isolated proteins may be obtained from various sources. They may be extracted or purified from natural sources, such as plants, animal milk, animal tissue, microorganism, etc. using known methods or they may be obtained commercially. Suitable proteins or protein compositions (i.e. mixtures of different types of proteins and/or proteins from different sources) include for example total milk proteins, individual milk proteins, such as one or more whey proteins, e.g. β-lactoglobulin, α-lactalbumin, bovine serum albumin, etc., and/or one or more caseins such as α-caseins, β-caseins, κ-caseins and γ-caseins or total caseins or total whey proteins. Total whey proteins can for example be obtained from Davisco Foods, USA (e.g. Bipro).

Other suitable protein sources are plant proteins, such as one or more (e.g. total) wheat proteins, soybean proteins, pea proteins, lupin proteins, canola or oilseeds rape proteins, maize proteins, rice proteins, and many others. Similarly, animal proteins such as gelatin, one or more blood proteins, one or more egg, meat or fish-proteins may be used. In one embodiment also microbial proteins such as one or more bacterial proteins and/or fungal proteins (including yeast proteins) are used. It is understood that also recombinantly produced proteins may be used, such as e.g. recombinantly produced lactoglobulin.

Especially preferred proteins for use in the method are one or more of whey protein isolate, whey protein concentrate, β-lactoglobulin and a mixture of β-lactoglobulin and α-lactalbumin.

The proteins preferably comprises at least about 1 cystein residue per protein or per peptide molecule, more preferably at least 2 cystein residues per protein or peptide molecule, even more preferably at least 3 cystein residues per protein or peptide molecule and most preferably at least 4 cystein residues per protein or peptide molecule. As mentioned, preferably full length proteins are used. Most preferably at least about 80%, 90%, more preferably at least 95% or 98% or more (especially 100%) of the proteins and/or peptides used fulfill the above requirements. Proteins and/or peptides lacking sufficient cystein residues may be removed prior to use. Similarly, protein hydrolysates which comprise insufficient proteins and/or peptides having the above mentioned number of cystein residues may be discarded or alternatively enriched for the appropriate proteins/peptides.

In one embodiment the proteins preferably comprises at least about 1 or even 2 cystein residues per 500, especially per 400 amino acids, more preferably at least 1 or even 2 cystein residues per 300 or 200 amino acids, even more preferably per 100, 30 or 20 amino acids. The average molecular weight of the protein is preferably at least 5, 10, 15, 20, 50, 100, 200, 250 or more kDa as determined by SDS-PAGE analysis.

When protein hydrolysates are used, the hydrolysis is preferably such that at least 20%, 30%, more preferably at least 40 or 50% (or more, e.g. 60, 70, 80 or 90%) of the protein fragments in the hydrolysate have a length of at least about 10, 20 or 30 amino acids or longer, such as 40, 50, 60 amino acids or more.

Depending on the type of encapsulated particle which is to be made, one or more additives may be added to (and mixed with) the aqueous protein solution either prior to protein activation (i.e. in step a.) and/or after protein activation, i.e. in step b) of the above method or may be added as such during spraying and/or drying. Additives that may be suitably added are described further below. In certain embodiments these additives include “sensitive additives”, which are additives that are preferably protected from exposure to external factors and are therefore preferably either incorporated in the protein coating itself and/or even more preferably beneath at least on coating layer (e.g. in or on the core material or the encapsulated fat).

### Step b)

In step b) of the method, the protein solution (which optionally further comprises additives) is submitted to a protein activation treatment. The nature of this treatment is not essential, as long as the protein becomes sufficiently activated for further use. Thus, although the activation treatment is preferably a heat treatment, other methods may also be suitable for achieving the same degree of protein activation, such as application of high pressure, shear forces, etc. Examples of suitable methods for achieving the same protein reactivity are microwave treatment, high pressure, shear, unfolding with urea, and combinations thereof. The skilled person can easily determine whether the treatment results in sufficiently activated (reactive) protein aggregates.

When heat treatment is used to activate the proteins, the temperature and time required for obtaining the minimum reactivity depends on the types of protein used and other conditions, such as pH of the solution, salts, etc. For example, heat treatment of a solution of 9% whey proteins (Bipro) in demineralized water for 7 minutes at 95° C. resulted in a reactivity of above 0.15 mM per 1 wt % protein solution (see Examples).

Heat treatment conditions, especially for generating protein encapsulated air particles, include for example the following: at least about 5, 6, 7, 8, 9 or 10 minutes at a temperature of at least about 90° C., 93° C., 95° C. or more. However, because many factors (type of protein, number of cystein residues, pH, etc.) affect the reactivity, a wide range of temperatures (e.g. 40-200° C.) and heating periods (ranging from seconds to hours) may be used to achieve a minimal reactivity. For any given type of protein and protein-comprising solution, the skilled person can easily define conditions which are suitable for obtaining the minimum reactivity required, without using undue experimentation. Any conditions which do not lead to protein aggregates having the required reactivity can then be disregarded and only optimal conditions used further.

### Reactivity

Whatever treatment is used for activation, the treatment should be sufficient to result in protein aggregates having a reactivity of at least 0.10 mM sulphydryl or thiol groups per 2 wt % protein solution, more preferably at least 0.2 mM, 0.3 mM or even more, such as 0.4 or 0.5 mM or more. For example, whey protein dissolved in water was found to reach sufficient reactivity when exposed to 95° C. for 7 minutes (see Examples), but other activation treatments may lead to the same reactivity.

Reactivity is required to covalently cross-link protein aggregates. The reactivity is defined as the number of thiol groups per amount of protein expressed as the concentration of thiol or sulfhydryl groups (mM) per 2 wt % protein solution. Exposure of thiol groups, which leads to their reactivity, can be achieved by e.g. heat-treatment.

### Ellman's Assay

Reactivity can be determined at pH 7 according to the Ellman's assay (Ellman, 1959 vide supra). In this assay the number of thiol groups is determined using ε(412 nm)=13,600 M⁻¹ cm⁻¹ for 2-nitro-5-mercaptobenzoic acid (DTNB) and expressed as the concentration thiol groups (mM) per gram of protein (aggregates). The absorbance is measured at 20-25° C. The value after 30 minutes of incubation with DTNB is taken to calculate the reactivity. The reactivity is expressed as the concentration of thiol or sulphydryl groups (in mM) in a 2 wt % protein solution. Hence, reactivity is determined after 30 minutes of incubation at 20-25° C. of a 2 wt % protein solution, using ε(412 nm)=13,600 M⁻¹ cm⁻¹ for 2-nitro-5-mercaptobenzoic acid (DTNB). Such an assay would typically be taken at pH 6-9.

A convenient way to perform the Ellman's assay is described in Alting et al. (Formation of disulphide bonds in acid-induced gel of preheated whey protein isolate. J. Agric. Food Chem. 48 (2000) 5001-5007). Typically, 0.25 ml of a 1 mg/ml DTNB solution in 50 mM imidazol-buffer pH 7 (pH adjusted with HCl), 0.2 mL protein solution (2 wt % protein solution) and 2.55 ml imidazol-buffer pH 7 are mixed. The assay is preferably performed in the absence of detergents such as urea or SDS.

Alternatively, step b) of the invention may be described as follows:
b1) submitting the aqueous solution provided in step a) to an activation treatment, to obtain protein aggregates;
b2) measuring the number of thiol groups of said protein aggregates using the aforementioned Ellman's assay; thus determining the reactivity, and
b3) selecting the activated protein aggregates having at least 0.10 mM sulphydryl or thiol groups per 2 wt % protein solution;

### Steps c) and d)

The aqueous solution comprising the reactive protein aggregates (and optionally other additives) may be used further in three embodiments of the invention, as depicted and discussed herein below.

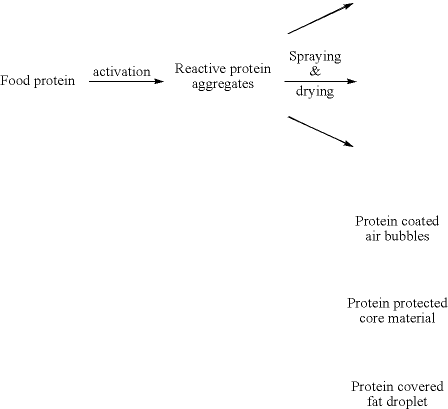

### I. Protein Encapsulated Air

In the first embodiment, the aqueous solution comprising the reactive protein is sprayed as such, or with certain additives, which are added either in step a) (i.e. before protein activation) and/or in step b) (after protein activation), to form particles. The activated protein is, however, not sprayed around a core, so that air is encapsulated within the protein coating. This is exemplified in Example 1 and FIGS. 1 and 2, wherein it is shown that very small, water-insoluble, uniform, stable protein particles are made, which enclose air.

Preferably, the coated air particles are obtained by spray-drying, resulting in a dried powder comprising the spherical particles. Spray-drying can be carried out as known in the art, for example as described in U.S. Pat. No. 6,223,455 or the “Spray Drying Handbook”, K. Masters, 5th ed., Longman Scientific & Technical Publishers, 1991, pp. 329-337 and 346-349. Averaged particle size and shape can be controlled by, for example, using nozzles of various sizes during the spray-drying step.

The volume weighted averaged particle size is typically within the range of 1-100 μm, especially within the range of 2-80 μm. According to a particularly preferred embodiment, the averaged particle size is equal to or below 50 μm in diameter, such as equal to or less than 25, 20, 15, 10 or 5 μm. Size and shape can be analyzed using microscopy (e.g. light microscopy or electron microscopy) or light scattering. Preferably at least 80%, 85%, 90% or more of the particles have a diameter of 50 μm or less, such as 40, 30, 25, 15, 10 μm or less.

One or more of the following (food-grade) additives may be added to the protein aggregates, either before the activation treatment (during step a.) and/or after the activation treatment (during or after step b.), but prior to and/or during spraying these to form particles:
polyols such as: glycerol, xylitol;
menthol, glyceryl triacetate, di-(2-ethylhexylexyl) adipate;
plasticizers, such as glycerol, glyceryl triacetate and/or di-(2-ethylhexylexyl) adipate, or others, or mixtures of two or more plasticizers; the addition of one or more plasticizers improves the flexibility of the protein coating; a preferred plasticizer is e.g. glycerol; the plasticizer is preferably added to the activated protein aggregates and mixed in an amount of 10 to 70 wt % on the protein basis, most preferably 20 to 40 wt %.
sugars such as for example: lactose, sucrose, glucose, galactose.
hydrolcolloids such as for example: gum Arabic, alginate, pectin, starch, xanthan, carrageenan, guar gum, locust bean gum, tara gum, gellan gum.
salts such as for example: sodium salts, calcium salts, potassium slats;
enzymes such as for example: proteases, peptidases, oxidases, hydrolases, esterases, lyases;
cross-linkers such as for example: tannins, transglutaminase, formaldehyde, glutaraldehyde,
   “sensitive additives” are additives which benefit from being protected from the environment (especially from the digestive tract or parts thereof, but also light, temperature, acids, radiation, etc.) and include e.g. flavours, salts, enzymes, microorganisms (e.g. bacteria such as one or more probiotic bacterial strains), prebiotics, peptides, minerals, vitamins, fatty acids (e.g. PUFAs), drugs, bioactive components, hormones, etc.

Preferably the additives are not reactive towards the activated protein aggregates, e.g. the additives do not react with free sulfhydryl (thiol) groups as this would interfere with the cross-linking of the protein in the subsequent spray step. The exception to this concerns cross-linkers which will assist in crosslinking the activated protein aggregates, hence cross-linkers preferably are susceptible to reaction with sulphur groups.

When one or more of the above additives are added to the aqueous solution prior to activation treatment, i.e. in step a. of the method, the conditions of step b. for generating activated protein aggregates may need to be modified or adjusted accordingly. When the solution is not sprayed onto a core, the additives, or at least a part thereof, will be incorporated into the protein coating, so that encapsulated air particles are formed which comprise one or more additives in the coating.

Optionally, one or more additives may be sprayed onto the dried particles, so that a layer outside the protein coating is formed. This layer may then again be covered by further coatings to form multi-layered encapsulated air particles.

Optionally, spray drying can occur in the presence of modified atmosphere, N₂, or other gas for additional protection of the sensitive ingredient.

Thus, the encapsulated air particles formed in this method may be used as such or they may be coated one or more additional times. For example, to add further coatings, the particles may be used as “core” material in the embodiment herein below. This way, multi-layered protein encapsulated-air particles can be made. Thus, additional layers of activated protein aggregates, layers of other hydrocolloids (such as gum arabic, layers of fat, carbohydrates, additives mentioned above, etc. may be added. At least one further layer is especially preferred if the protein particles comprise sensitive additives in the primary protein coating.

It is not necessary to use the method of the invention for applying one or more further layers onto the dried particles, although this is possible for further layers comprising reactive protein aggregates. Thus, any suitable coating method may be used for the addition of further layers.

### II. Protein Encapsulated Core Material

In another embodiment core material is coated with activated protein aggregates. Optionally, additional components (additives, including sensitive additives) are present on or within the core material. Such components are for example enzymes, prebiotics, probiotics, or other components which benefit from being protected by a protein coating. They are herein referred to as “sensitive components” or “sensitive additives” (see above).

The additives may be added to the protein solution of step a) or of step b), i.e. either before protein activation or after protein activation, but before or during spraying and drying. The additives may also be applied during spraying and/or drying as such, e.g. without adding them to the protein solution.

In this method, step c) comprises spraying the solution (comprising the activated protein aggregates and optionally one or more additives) onto a core material to form protein coated core particles.

Various core materials may be used. For example, the core material may comprise or consist of hydrocolloids (e.g. carboxymethylcellulose, starch, maltodextrin) and/or fat and/or wax and/or carbohydrates (e.g. sugars). The core particles are preferably spherical. Suitable core particles include particles, preferably round, of at least about 10, 20, 30, 40, 50, 60, 70, 80, 90, 100 μm or more, such as 200, 300, 350, 400, 500, 600, 700, 800, 900, 1000, 2000, 3000, 4000, 5000 μm diameter. Such core particles can be obtained commercially or prepared separately. In one embodiment, the encapsulated air particles described above are used as “core material”.

Preferably, the activated protein aggregate solution is sprayed and dried using e.g. fluidized bed or spouted bed equipment. Such equipment is available in the art, see e.g. Fluid bed coater GPCG 1.1 with Wurster insert (Glatt GmbH).

One or more of the sensitive additives can be buried within the core particle made by e.g. extrusion or other technique.

Preferably the sensitive component(s) are either buried within the core material or coated onto the core material. In one embodiment they may, however, also be added to the activated protein aggregate itself, prior to (or during) spraying or to the aqueous solution of step a) prior to protein activation.

The sensitive additives are preferably one or more components selected from the group consisting of: an enzyme, a probiotic (preferably a live or viable bacterium), a prebiotic, a vitamin, a polyunsaturated fatty acid (PUFA), a flavour (e.g. a bitter component, a salty component, an acid components, etc.). However, this list is non-limiting, as any component, preferably food-grade, which benefits from protection against the environment, such as oxygen, moisture, acid conditions, interaction with food matrix, temperature, any part of the intestinal tract environment (e.g. mouth/saliva, stomach acids, intestine, etc.) etc. may be used.

Optionally one or more further layers of activated protein aggregate and/or additives (e.g. sensitive ingredients), and/or other layers (such as layers of other additives, layers of other hydrocolloids e.g. gum arabic; carbohydrate solutions; fats; food colourings, etc.) can be sprayed onto the coated particles to create multi layered particle. Such a particle may, for example comprise the following layers, from inside to outside: core matrix (optionally comprising sensitive component 1), protein coating (primary layer/particles), sensitive component 2 (second layer), protein coating (third layer), sensitive component 3 (fourth layer), protein coating (fifth layer).

Such single or multi-layered particles may have various final diameters, such as 50 μm, 100 μm, 250 μm, 500 μm, 1 mm, 2 mm, 3 mm, 4 mm, 5 mm, 6 mm or more. Typically, these particles have a volume weighted averaged diameter in the range of 100 μm to 5 mm, especially in the range of 200 μm-2 mm.

In a further preferred embodiment the particles are acid resistant, i.e. the particles remain intact in the stomach and the sensitive components are only released when contacted with enzymes secreted into the lower intestinal tract, such as pancreatic enzymes. This is especially achieved by at least one or multiple layers as described, preferably by at least one strong layer of activated protein aggregates surrounding the sensitive additives.

In one embodiment also one or more further additives (e.g. fats, hydrocolloids, carbohydrates, enzymes, micro-organisms, etc) may be added to the protein aggregates (either before protein activation and/or after protein activation) prior to, or during, spraying these to form particles, as described herein above for encapsulated air particles. In addition, further additives (such as fats, hydrocolloids, carbohydrates) and/or other additives (e.g. sensitive ingredients, such as enzymes, microorganisms) can be layered onto the particles by spraying.

### III. Protein Encapsulated Fat

In another embodiment fat or a fat-comprising solution is mixed prior or after activation treatment, before spraying step c), with the protein material, to form an oil-in-water or a water-in-oil emulsion. Preferably, the fat or fat-comprising solution is added to the solution comprising the activated protein aggregate.

Any fat may be suitable, in particular food-grade fats, such as plant derived oil (e.g. sunflower oil, canola oil, palm oil, soybean oil, flax oil, safflower oil, peanut oil, maize oil, olive oil, pumpkin oil, etc.). Especially oils and fats rich in poly unsaturated fatty acids (PUFA) may be used. In one embodiment fats or oils comprising or consisting of omega-3 and/or omega-6 fatty acids are used. Oil rich in omega-3 and/or omega-6 can be derived from various sources, such as marine sources (marine algae, fish oil) or non-marine sources (e.g. plants such as flax or canola, or transgenic plants, or microbial produced). Also animal-derived fats and oils may be used, such as milk fat or fish oil. Likewise, microbial produced fats and oils can be used.

The fat or fat-comprising solution is preferably mixed with the aqueous solution comprising the activated protein aggregates and the mixture is homogenized using known methods. The ratio (w/w) of protein to fat is comprised between 0.04:1 and 4:1, preferably 0.4:1,

The homogenate is then sprayed to form particles and these are dried. Preferably, spray-drying is used, so that a powder comprising the particles is obtained.

The volume weighted averaged particle size is typically within the range of 1-100 μm, especially within the range of 2-80 μm. According to a particularly preferred embodiment, the average particle size is equal to or below 50 μm, such as equal to or less than 25, 20, 15, 10 or 5 μm. Size and shape can be analyzed using microscopy (e.g. light microscopy or electron microscopy) or light scattering. Preferably at least 80%, 85%, 90% or more of the particles have a diameter of 50 μm or less, such as 40, 30, 25, 15, 10 or less.

In one embodiment also one or more additives may be added to the protein aggregates (either before protein activation and/or after protein activation), or to the fat (or fat-comprising solution), or to the (pre)emulsion prior to (or during) spraying the emulsion to form particles, as described herein above for encapsulated air particles and for encapsulated core particles.

One or more further layers (also as described in the other embodiments above) may be added (e.g. sprayed) around the protein encapsulated fat particles, using e.g. any spraying and drying method.

Protein Encapsulated Particles, and Food/Feed, Cosmetic or Pharmaceutical Compositions Comprising these

Independent of the selected encapsulation embodiment I-III, the activation treatment and reactivity assay in step b) of the method of the present invention provide means for controlling the water-solubility of the particles to any extent desired, thus making it possible to distinguish from untreated reference material exhibiting 100% water-solubility (see examples). However, for many applications it is preferred that the particles are water-insoluble.

According to a particularly preferred embodiment, the protein particles are not soluble in water as evidenced by the fact that less than 20 wt. % of the particles dissolves when 1 g of the protein encapsulated particles is dispersed in 100 ml of distilled water of 20° C. under continuous stirring for 10 minutes. According to an even more preferred embodiment the particles are not soluble under conditions such as those prevailing in the human stomach. Thus, most preferably, less than 20 wt. % of the particles dissolves when 1 g of the protein encapsulated particles is dispersed in 100 ml of water with pH 3.0 under continuous stirring for 10 minutes, said water having a temperature of 37° C. and having been prepared by adding HCl to distilled water. Naturally, the stirring conditions employed in the above tests should be gentle, i.e. sufficient to disperse the particles and not to mechanically break up the protein encapsulated particles.

Further, the protein coating is stable, preferably sufficiently stable to remain intact when dispersed in water using standard techniques. Standard protein determination assays can be applied to check the solubility/stability.

Advantageously, a substantial fraction of the cystein residues in the cross-linked protein is actually participating in disulphide cross-links, i.e. in the cystein-cystein cross-links. The unique properties of the present protein encapsulated particles are particularly evident in case the cross-linked protein contains a high level of disulphide cross-links, i.e. disulphide cross-links that have been formed as a result of reactions between reactive cysteine residues in the proteins.

According to a preferred embodiment, the encapsulation matrix contains at least 60 wt. %, most preferably at least 80 wt. % of the disulphide cross-linked protein. The encapsulation matrix typically represents up to 100 wt. % of the protein encapsulated particles. For instance, in case the encapsulation matrix is used to encapsulate air bubbles, said matrix can represent 100 wt. % of the particles. In case the encapsulation matrix is used to, for instance, encapsulate core particles, notably core particles of a large diameter, said encapsulation matrix may suitably represent not more than 5 wt. % of the protein encapsulated particle, or even less if such a particle has been provided with an additional external coating. (e.g. a high melting wax of fat coating). Accordingly, in a preferred embodiment, the encapsulation matrix represents 3-100 wt % of the protein encapsulated particles. Preferably, the encapsulation matrix represents 10-99 wt. %, most preferably 20-99 wt. % of the protein encapsulated particles.

The benefits of the present invention are particularly pronounced in case a cross-linked protein is employed that is selected from the group consisting of whey proteins, egg proteins, soy protein, and combinations thereof. Most preferably, the cross-linked protein is a whey protein.

The particle size of the protein encapsulated particles of the present invention may vary widely. Typically, the particles have a volume weighted average diameter of 1 μm to 5 mm. The volume weighted average diameter is suitably determined with the help of a set of sieves with different mesh sized or by light scattering.

One embodiment of the present invention relates to protein encapsulated particles containing a core particle with a diameter of at least 10 μm, said core particle being enveloped by the encapsulation matrix. The manufacture of these types of encapsulated particles has been described herein before. Typically, the present encapsulation matrix represents between 1 and 80 wt. %, preferably between 5 and 60 wt. % of these encapsulate particles.

Another embodiment of the invention, which has also been described above, relates to protein encapsulated particles containing at least 1 wt. % of fat globules, said fat globules being enveloped by the encapsulation matrix. Preferably, the protein encapsulated particles contain between 5 and 30 wt. % of the fat globules. The fat globules typically have a volume weighted mean diameter in the range of 0.2-10 μm, especially of 0.4-5 μm.

Yet another embodiment concerns protein encapsulated particles containing at least 10 vol % of air bubbles, said air bubbles being enveloped by the encapsulation matrix. Typically, the amount of air bubbles contained in the encapsulated particles is within the range of 12.-60 vol. %.

The protein encapsulated particles of the present invention may advantageously be employed as a vehicle for delivering biologically active ingredients to an animal or a human. In particular protein encapsulated particles that are stable under gastric conditions may suitably be used to deliver biologically active ingredients that are not stable under gastric conditions. Thus, one aspect of the invention relates to the of use the present protein encapsulated particles in therapeutic or prophylactic treatment, said treatment comprising oral administration of the protein encapsulated particles. Typically, the protein encapsulated particles are orally administered in an amount of 0.1 to 40 g per administration event. In accordance with this aspect of the invention, the biologically active ingredient may be a pharmaceutically active ingredient or a nutrient (including micronutrients such as vitamins).

The protein encapsulated particles, obtainable by any one of the above methods, or mixtures of such particles, are also provided as one embodiment of the invention. For example powders, gels, or capsules comprising one or more types of particles are provided. These can be ingested as such, or they can be used as ingredients for food or feed products, or for cosmetic or pharmaceutical products. Powders comprising or consisting of the particles may for example be added to (warm or cold) liquids, either alone or together with other ingredients.

Thus, also food or feed products, cosmetic or pharmaceutical products, comprising one or more of the particles are provided. For example, a food product may comprise both protein encapsulate air particles, as well as protein encapsulated core particles and/or protein encapsulated fat as described above.

The particles may be added during the production process of the food/feed, cosmetic or pharmaceutical product and/or to the final food/feed, cosmetic or pharmaceutical product.

Food products comprising the particles include for example the following: cold or warm drinks, such as coffee, chocolate, tea, fruit or vegetable juices; soups; sauces; spreads, batters, ready-to-eat meals, dairy products (milk, milk-based drinks, yoghurt, cheese, butter, margarine, ice cream), pasta, fruit or vegetable products, meat or fish products, meat replacers, bread, pastries, deserts, sweets, candy-bars, confectionary, food- or drink-additives (such as coffee or tea creamers, sweeteners), powders such as instant coffee or tea, milk-powder, soup powder, ice-cream, etc.

Feed products include any type of animal feed, such as feed for farm animals (cows, horses, pigs, chicken, etc.), pets (dogs, birds, fish, cats, rabbits, rodents, etc), wild animals, etc.

Suitable amounts of the particles may vary, depending on the product. However, preferably such a product comprises at least 0.001 wt % of particles, such as at least 0.5 wt % to 100 wt % of particles, more preferably at least about 1 wt %, 2 wt %, 5 wt %, 10 wt % or more. For example, an instant coffee powder or coffee creamer may comprise between 0.5 wt % and 5 wt % particles or particle mixtures.

### DESCRIPTION OF THE FIGURES

FIG. 1—shows a microscopic image of encapsulated air particles produced as described in Example 1.

FIG. 2—an SDS-agarose gel is shown on which in the two lanes dispersions of reactive protein aggregates were applied to the gel (on the top of the gel); samples before (left lane) and after (right lane) spray-drying are shown.

### EXAMPLES

### Example 1

### Protein Coated Air

### 1.1 Preparation of Aggregates

Whey protein isolate (Bipro, Davisco, USA) was dissolved in water at a concentration of 9% protein (w/w). This solution was subsequently heated (under shear) for 5 minutes at 95° C. After this the solution was rapidly cooled to room temperature.

The reactivity of the particles was determined using Ellman's assay as described before. The reactivity was 0.23 mM per 1% protein solution.

### 1.2 Spray-Drying to Form Particles

In the next step the protein solution was spray-dried using a NIRO 25 spray-dryer. By spray-drying a powder was obtained.

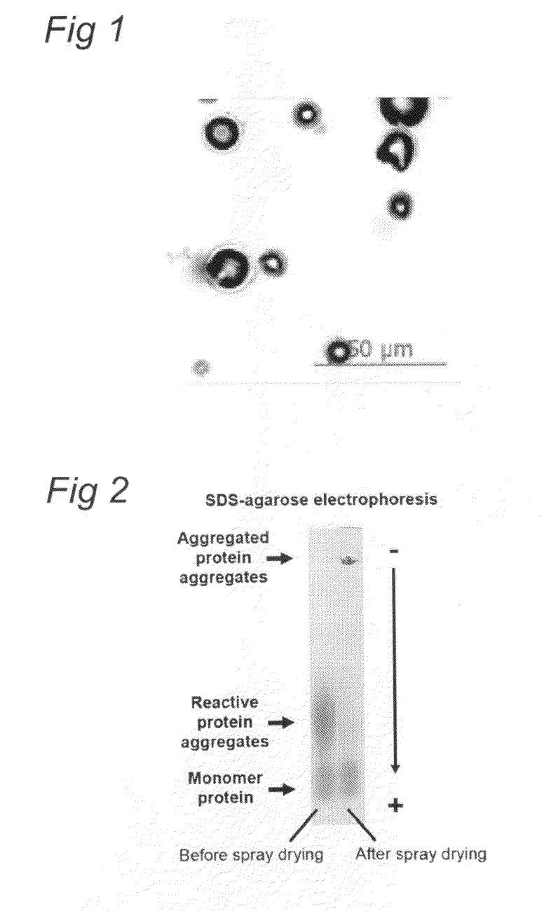

This powder was analyzed by microscopy and SDS-agarose electrophoresis (according to Alting et al. (2000) J. Agric food Chem. 48, 5001-5007). The microscopic image is shown in FIG. 1, which shows particle size and shape.

FIG. 2 shows that the covalent cross-linking of reactive protein aggregates has occurred. It is clear that before spray-drying, in addition to non-aggregated monomer protein clearly a band corresponding to reactive protein aggregates is present. After spray-drying the band corresponding to reactive protein aggregates has disappeared and a new band on top of the gel, corresponding to very large protein material that can not enter the electrophoresis gel-network, has appeared. This unambiguously shows that during spray-drying cross-linking between reactive protein aggregates, likely via disulphide bonds, has occurred, resulting in an increased water-insolubility of the protein matrix.

The stability of the protein-coated air-bubbles was tested by dispersing them in water. It was observed that even after incubation for hours the particles did not solubilize.

### Example 2

### Protein Protected Core Material

### 2.1—Encapsulation of Enzymes

Reactive protein aggregates were prepared by heating a 9% w/w whey protein isolate solution (Bipro; Davisco, USA) in demineralized water (under shear) for 5 minutes at 95° C. After cooling, glycerol (20% based on protein content) is mixed to the protein mixture. The reactivity of the particles was determined using the method as described by Alting et al (2000). The reactivity was 0.23 mM per 1% protein solution.

To the reactive protein aggregates amylase was added (1% on protein base m/m). The reactive protein aggregates/enzyme mixture is then sprayed using a fluidized bed coater (Glatt, Germany) onto methylcellulose round core material (Cellets, Syntapharm, Germany) with a diameter size of 350 μm. Afterwards, an extra layer of gum arabic (20% w/w solution) is sprayed onto the capsules. The capsules are then dispersed in different buffers and visualized under the microscope. Under stomach conditions, the layer around the capsules remained intact. Under gut conditions (in the presence of pancreatine), the layer of protein and gum arabic slowly fell apart and released the enzyme.

The amylase activity is greater when the enzyme is encapsulated than when it is not encapsulated, showing the efficiency of targeted delivery in the gastrointestinal tract using the protein-based coating.

### 2.2—Encapsulation of Probiotics

Reactive protein aggregates were prepared by heating a 9% w/w whey protein isolate solution (Bipro; Davisco, USA) in demineralized water heated (under shear) for 7 minutes at 95° C. After cooling, glycerol (20% based on protein content) is mixed to the protein mixture. The reactivity of the particles was determined using the DTNB-method as described before. The reactivity was above 0.1 mM/2% protein solution.

The probiotic powder was dispersed in a polymer mixture and extruded, followed by a spheronization step. The protein/glycerol mixture is then sprayed using a fluidized bed coater (Glatt, Germany) onto the round extruded core material comprising the probiotics. An extra fat layer was applied with the fluidized bed coater.

The capsules are then tested under stomach conditions and the survival of the encapsulated bacteria was higher than the non encapsulated bacteria.

### Example 3

### Protein Covered Fat Droplet

### 3.1—Encapsulation of Fat-Soluble Compounds

Reactive protein aggregates are prepared by heating a 9% w/w whey protein isolate solution (Bipro; Davisco, USA) in demineralized water (retort) for 2 h at 68.5° C. After cooling, glycerol (20% based on protein content) is mixed to the protein mixture. The reactivity of the particles is determined using the DTNB-method as described before. The reactivity was 0.17 mM per 1% protein solution.

A pre-emulsion is prepared by mixing reactive protein aggregates (5% protein w/w) and sunflower oil (30%) using an Ultra-turrax. The mixture is then homogenized using a two-stage high pressure homogenizer (Niro saovi lab-scale; flow 10 L/h) at a temperature of 60° C. (200/20 bar).

In the next step the emulsion is spray-dried using a Buchi lab-scale spray-dryer. By spray-drying a powder is obtained.

### Example 4

### Blocking Thiol Groups Prevents Disulphide Cross-Linking

### 4.1—Preparation of Particles and Blocking of Thiol Groups

Whey proteins isolate (Bipro, Davisco, USA) was dissolved in water at a concentration of 9% protein (w/w). This solution was subsequently heated (retort) for 2 h at 68.5° C. The solution was then rapidly cooled to room temperature. To the solution, 5 mM (final concentration) N-ethylmaleimide was added to chemically block the thiol-groups.

The blocking of the thiol groups (reactive groups of the particles) was determined using the DTNB-method as described before. The reactivity was below 0.03 mM per 1% protein solution.

### 4.2 Spray-Drying to Form Particles

In the next step the protein solution was spray-dried using a small scale spray dryer (Büichi). By spray-drying a powder was obtained.

The stability of the protein coated particles was tested by dispersing them in water. It was observed that the particles easily solubilize within hours.

This example unambiguously shows that during spray-drying disulphide crosslinking between reactive protein aggregates was prohibited, resulting in a water-soluble protein matrix. Disulphide cross-linking during spray drying is a pre-requisite to obtain water-insoluble protein matrix.

### Example 5

### Preparation of Whey Protein Solutions

Various whey proteins solutions were prepared. For all solutions, whey protein isolate (Bipro, Davisco, USA) was dissolved in water at a concentration of 9% protein (w/w). This solution corresponds to the “native solution” (Native). The WPI native solution was subsequently heated under different conditions (three different heating times and temperatures) as described in Table 1. After this the solution was rapidly cooled to room temperature.

TABLE 1
Various types of WPI heated solutions versus native solution

| | | | Type of | Heating | Reactivity* (mM |
|---|---|---|---|---|---|
| Name | Heating time | | heating | temperatures | per 1% protein) |
| Agg 1 | 2 | hours | retort | 68.5° C. | 0.17 |
| Agg 2 | 30 | minutes | retort | 90.0° C. | 0.20 |
| Agg 3 | 5 | minutes | shear | 95.0° C. | 0.23 |
| Agg 4 | 7 | minutes | shear | 90.0° C. | 0.18 |

| | | | | |
|---|---|---|---|---|
| Native | — | — | — | 0.018 |

*The reactivity of the particles (Agg 1, Agg 2, Agg 3 and Agg 4) was determined using Ellman's essay as described before.

### Example 6

### Preparation of Encapsulates with a Hard Core

Whey protein solutions are prepared as described in Example 5. The reactive protein aggregates mixtures (Agg 1, Agg 2, Agg 3) were sprayed using a fluidized bed coater (Glatt, Germany) onto methylcellulose round core material (Cellets®, Syntapharm, Germany) with a diameter size between 350 and 500 μm.

The native protein solution (Native) was sprayed using a fluidized bed coater (Glatt, Germany) onto methylcellulose round core material (Cellets®), Syntapharm, Germany) with a diameter size of 350 μm.

The preparation recipe of the resulting encapsulates is summarized in table 2.

TABLE 2
Overview of encapsulates prepared with the fluidized bed coater

| Name encapsulates | Core | Coating |
|---|---|---|
| Encaps 1 | Cellets ® | Agg 1 |
| Encaps 2 | Cellets ® | Agg 2 |
| Encaps 3 | Cellets ® | Agg 3 |
| Encaps 4 | Cellets ® | Agg 4 |
| Encaps N | Cellets ® | Native |

### Example 7

### Solubility Assay of Encapsulates with a Hard Core

Encapsulates were prepared as described in example 6. The solubility of the coating was tested at pH 2 at 37° C. and at pH 7 at 20° C. The encapsulates (Encaps 1, Encaps 2, Encaps 3, Encaps N) were added to deionized water in order to obtain a concentration of total protein of 500 μg/ml. The pH was then adjusted to the desired value. The encapsulates were gently stirred overnight. The supernatant was filtered and colored with BSA protein essay kit. The soluble proteins were quantified by spectrophotometer reading at 562 nm.

As shown in table 3, both at pH 2 and pH 7, the encapsulates prepared with reactive WPI solution (Encaps 1, Encaps 2, Encaps 3) were less soluble than the encapsulated prepared with native WPI solution (Encaps N). It further shows that the solubility can be modulated by the preparation of reactive WPI solutions, i.e. heat treatment.

TABLE 3
Water-solubility of encapsulates

| | Solubility (%) | Solubility (%) |
|---|---|---|
| Encapsulate | at pH 2 | at pH 7 |
| Encaps N | 100 | 100 |
| Encaps 1 | 59 | 74 |
| Encaps 2 | 24 | 28 |
| Encaps 3 | 23 | 24 |

### Example 8

### Survival of Encapsulates Under Gastric Conditions

Encapsulates were prepared as described in example 6. The encapsulates survival under gastric conditions was tested by adding the encapsulates (Encaps 1, Encaps 2, Encaps 3, Encaps N) in a mixture of gastric juice (i.e. 20 mg/g pepsin, pH controlled to 2.0, 37° C.). The encapsulates were gently stirred during 2 hours. Samples were regularly taken at relevant times (i.e. 0 min, 15 min, 30 min, 60 min, 120 min) and the pepsin activity was directly inactivated by increasing the pH to 7.0. The amount of degraded protein was measured by determination of free NH₂ groups using the OPA method.

It was observed that the percentage of hydrolyzed protein (i.e. ‘broken’ coating) increased in time. While the increase was instantaneous for encapsulates coated with native WPI (Encaps N), a gradual and slow increase was observed for the encapsulates coated with reactive WPI (Encaps 1, Encaps 2, Encaps 4).

### Example 9

### Preparation of Encapsulates with Fat

Whey protein solutions were prepared as described in Example 5.

A pre-emulsion was prepared by mixing 277.8 g of reactive protein aggregates suspension (Agg 1, Agg 2, Agg 3) with 69.5 g of sunflower oil and 152.8 g of demineralized water using an Ultra-turrax. The mixture was then homogenized using a two-stage high pressure homogenizer (Niro saovi lab-scale; flow 10 L/h) (180/30 bar). In the next step the emulsion was spray-dried using a Buchi lab-scale spray-dryer (inlet temperature: 152° C., outlet temperature: 90° C.). By spray-drying a powder was obtained.

The control corresponds to a pre-emulsion prepared by mixing native protein solutions instead of reactive protein aggregates. The same conditions as described above were used to prepare encapsulated fat.

The resulting encapsulates are summarized in Table 4.

TABLE 4
Overview of encapsulates prepared with the spray dryer

| | Ingredient | |
|---|---|---|
| Name encapsulates | encapsulated | Coating |
| Encaps 5 | Sunflower oil | Agg 1 |
| Encaps 6 | Sunflower oil | Agg 2 |
| Encaps 7 | Sunflower oil | Agg 3 |
| Encaps N2 | Sunflower oil | Native |

### Example 10

### Solubility of Encapsulates with Fat

Encapsulates were prepared as described in example 9.

The solubility of the coating was tested at pH 2 at 37° C. and at pH 7 at 20° C., and the results shown in table 5. The encapsulates (Encaps 5, Encaps 6, Encaps 7, Encaps N2) were added to deionized water in order to obtain a concentration of total protein of 500 μg/ml. The pH was then adjusted to the desired value. The encapsulates were gently stirred overnight. The supernatant was filtered and colored with BSA protein essay kit. The soluble proteins were quantified by spectrophotometer reading at 562 nm.

At pH 2, the encapsulates prepared with reactive WPI solution (Encaps 5, Encaps 6, Encaps 7) were less soluble than the encapsulated prepared with native WPI solution (Encaps N2). At pH 7, the encapsulates Encaps 6 and Encaps 7 were less soluble than the encapsulated prepared with native WPI solution (Encaps N2) and Encaps 5.

Furthermore, the coating solubility varied for Encaps 5, Encaps 6 and Encaps 7, giving proof that the solubility can be modulated by the preparation of reactive WPI solutions, i.e. heat treatment.

TABLE 5
Water-solubility of encapsulates

| | Solubility (%) | Solubility (%) |
|---|---|---|
| Encapsulates | at pH 2 | at pH 7 |
| Encaps N2 | 100 | 100 |
| Encaps 5 | 36 | 70 |
| Encaps 6 | 4 | 7 |
| Encaps 7 | 19 | 24 |

### Example 11

### Solubility in the Presence of NEM

Encap 5 was prepared as described in example 6.

The solubility of the coating was tested at pH 7 at 20° C. in the presence of NEM. The encapsulate Encaps 5 was added to deionized water with 5 mM NEM at concentration of total protein of 500 μg/ml. The encapsulate was gently stirred overnight. The supernatant was filtered and colored with BSA protein essay kit. The percentage soluble protein was quantified by spectrophotometer reading at 562 nm.

The results showed that the solubility decreased to 15% compared to 71% when no NEM was added.

### Example 12

### Solubility in the Presence of Cu²⁺

Encaps N, Encaps 1 and Encaps 2 were prepared as described in example 6.

Their solubility was tested at pH 7 at 20° C. in the presence of Cu²⁺, and the results shown in table 6. The encapsulates were added to deionized water with 12 mM Cu²⁺ at a concentration of total protein of 500 μg/ml. The encapsulate was gently stirred overnight. The supernatant was filtered and colored with BSA protein essay kit. The percentage soluble protein was quantified by spectrophotometer reading at 562 nm.

For Encaps 1 and 2, the solubility decreased dramatically to <2%. Encaps N was completely soluble as compared to water without Cu²⁺.

TABLE 6
Water-solubility of encapsulates

| Encapsulates | Solubility (%) at pH 7 |
|---|---|
| Encaps N | 100 |
| Encaps 1 | 1 |
| Encaps 2 | 1 |

1.-24. (canceled)

25. A method for the production of protein encapsulated particles, said method comprising:
a. providing an aqueous solution comprising protein,
b. submitting said aqueous solution to a protein activation treatment to obtain activated protein aggregates having a reactivity of at least 0.10 mM thiol or sulphydryl groups per 2 wt % protein solution, as determined using Ellman's assay,
c. spraying said solution, comprising said activated protein aggregates, to form particles, and
d. drying said particles.

26. The method according to claim 25, wherein said protein is food-grade.

27. The method according to claim 25, wherein said protein aggregates have a reactivity of at least 0.2 mM thiol or sulphydryl per 2 wt % protein solution.

28. The method according to claim 27, wherein said protein aggregates have a reactivity of at least 0.3 mM thiol or sulphydryl per 2 wt % protein solution.

29. The method according to claim 25, wherein reactivity is determined after 30 minutes of incubation at 20-25° C. of a 2 wt % protein solution, using ε(412 nm)=13,600 M⁻¹ cm⁻¹ for 2-nitro-5-mercaptobenzoic acid (DTNB).

30. The method according to claim 25, wherein said particles contain air.

31. The method according to claim 25, wherein said solution is sprayed onto a core material to form a protein coated core particle.

32. The method according to claim 31, wherein said core material comprises proteins, hydrocolloids, carbohydrates, fat and/or wax.

33. The method according to claim 32, wherein said core further comprises one or more sensitive additives.

34. The method according to claim 33, wherein the one or more sensitive additives are selected from the group consisting of enzymes, micro-organisms, probiotic bacteria, prebiotics, vitamins, minerals, proteins, carbohydrates, peptides, polyphenols, fatty acids, drugs, bioactive components, polyunsaturated fatty acids (PUFA) and flavors.

35. The method according to claim 25, wherein said solution comprising activated protein aggregates is mixed with oil or fat to form an emulsion solution.

36. The method according to claim 25, wherein said protein is selected from the group consisting of milk proteins, whey proteins, caseins, protein hydrolysates, plant proteins, animal proteins, microbial proteins, and egg proteins.

37. The method according to claim 36, wherein said protein is selected from the group consisting of whey protein isolate, whey protein concentrate, β-lactoglobulin and a mixture of β-lactoglobulin and α-lactalbumin.

38. The method according to claim 25, wherein said protein comprises at least one cystein residue per protein or peptide molecule.

39. The method according to claim 25, wherein one or more additives are added to the solution obtained in step a. and/or in step b. prior to or during spraying, wherein said additives are selected from the group consisting of polyols, plasticizers, sugars, hydrocolloids, cross-linkers, fats and oils, sensitive additives probiotic bacteria, prebiotics, vitamins, minerals, proteins, carbohydrates, peptides, polyphenols, fatty acids, drugs, bioactive components, polyunsaturated fatty acids (PUFA) and flavors.

40. The method according to claim 39, wherein the sensitive additive is a micro organism.

41. The method according to claim 25, further comprising spraying one or more further layers around the particles obtained in step d).

42. A method for the production of protein encapsulated particles, said method comprising:
a. providing an aqueous solution comprising protein;
b. submitting said aqueous solution to an activation treatment, to obtain protein aggregates;
c. measuring the number of thiol groups of said protein aggregates using Ellman's assay;
d. selecting protein aggregates having at least 0.10 mM sulphydryl or thiol groups per 2 wt % protein solution from the activated aqueous solution;
e. spraying the activiated aqueous solution comprising activated protein aggregates, to form particles; and
f. drying said particles.

43. Protein encapsulated particles, obtainable by:
a. providing an aqueous solution comprising protein,
b. submitting said aqueous solution to a protein activation treatment to obtain activated protein aggregates having a reactivity of at least 0.10 mM thiol or sulphydryl groups per 2 wt % protein solution, as determined using Ellman's assay,
c. spraying said solution, comprising said activated protein aggregates, to form particles, and
d. drying said particles.

44. The protein encapsulated particles according to claim 43, wherein less than 50 wt % of the protein particles is soluble in water at 37° C. at pH 2 under continuous stirring for 10 minutes.

45. The protein encapsulated particles according to claim 43, said particles having a volume weighted averaged diameter of 1 μm to 5 mm.

46. The protein encapsulated particles according to claim 43, wherein the particles contain a core particle with a diameter of at least 10 μm.

47. The protein encapsulated particles according to claim 43, wherein the particles contain at least 10 vol. % of air bubbles.

48. A food, feed, cosmetic or pharmaceutical product comprising protein encapsulated particles according to claim 43.

49. The food product according to claim 48, wherein said food product is selected from the group consisting of a drink, a dairy product, a soup, a sauce, a desert, a candy-bar, a ice-cream, a food- or drink-additive, a ready-to-eat meal.

50. The food product according to claim 48, comprising at least 0.001 wt % of the protein encapsulated particles.

51. A method of therapeutic or prophylactic treatment of a condition, comprising orally administering the protein encapsulated particles according to claim 42.
